(19) United States
(12) Patent Application Publication
(10) Pub. No.: US 2013/0144190 A1
(43) Pub. Date: Jun. 6, 2013

(54) SLEEP APNEA DETECTION SYSTEM

(71) Applicants: Charles J. Bruce, Rochester, MN (US); Paul A. Friedman, Rochester, MN (US); Kevin E. Bennet, Rochester, MN (US); Virend K. Somers, Rochester, MN (US)

(72) Inventors: Charles J. Bruce, Rochester, MN (US); Paul A. Friedman, Rochester, MN (US); Kevin E. Bennet, Rochester, MN (US); Virend K. Somers, Rochester, MN (US)

(73) Assignee: MAYO FOUNDATION FOR MEDICAL EDUCATION AND RESEARCH, Rochester, MN (US)

(21) Appl. No.: 13/700,702

(22) PCT Filed: May 27, 2011
(86) PCT No.: PCT/US11/38397

§ 371 Date: Feb. 7, 2013

Related U.S. Application Data
(60) Provisional application No. 61/349,637, filed on May 28, 2010.

(51) Int. Cl. A61B 5/00; A61B 5/145; A61B 5/08; A61B 5/0205; A61B 5/0402; A61B 5/087; A61B 5/01; A61B 5/053; A61B 7/00; A61B 5/024

(52) U.S. Cl. CPC A61B 5/4818; A61B 7/003; A61B 5/7282; A61B 5/7257; A61B 5/0077; A61B 5/14542; A61B 5/0816; A61B 5/024; A61B 5/0402; A61B 5/087; A61B 5/082; A61B 5/01; A61B 5/053; A61B 5/02055; A61B 5/6898; A61B 5/0002
USPC 600/586

(57) ABSTRACT

This document provides methods and materials (e.g., systems) related to assessing sleep conditions (e.g., sleep apnea).

### CROSS-REFERENCE TO RELATED APPLICATIONS

This application claims the benefit of U.S. Provisional Application Ser. No. 61/349,637, filed May 28, 2010. The disclosure of the prior application is considered part of (and is incorporated by reference in) the disclosure of this application.

### TECHNICAL FIELD

This document relates to systems for detecting sleep conditions (e.g., sleep apnea).

### BACKGROUND

Monitoring of sleep quality and snoring are important concerns both from medical and personal perspectives. Many individuals are concerned as to whether they snore, whether they move around frequently during sleep, whether they sleep walk, whether they talk in their sleep, whether they grind their teeth (bruxism), and whether they have sleep apnea. These possibilities are often raised, particularly when people suffer from fatigue. Moreover, obstructive sleep apnea (OSA) is a medical condition in which intermittent airway obstruction (typically with manifest snoring) leads to hypoxemia, adrenergic discharge, hypertension, and an inflammatory state. OSA is frequently undiagnosed. However, determining whether an individual is at risk for or has OSA is important, since it is associated with hypertension, stroke, arrhythmias, and other significant maladies. Also a concern is obtaining objective assessment of sleep durations. Many patients complain of sleeping only very few hours a night, although objective assessment in a sleep lab or by actigraphy, may provide different conclusions.

### SUMMARY

This document provides methods and materials (e.g., systems) related to assessing sleep conditions (e.g., sleep apnea). The difficulty with sleep monitoring relates to billed expense and the delay between monitoring and access to data, cost of facilities and equipment, and the limited duration for which monitoring can be conducted, since a single night of sleep study may not reflect usual conditions, especially if the sleep study is done outside the home. Single polysonographys (PSGs) can correlate poorly with a repeat measurement. In fact, recent data confirm the poor reproducibility of PSG alone. The methods and materials provided herein can provide less expensive and easy options for multiple repeated measures of sleep conditions (e.g., sleep apnea severity) in the normal sleep environment.

In general, one aspect of this document features a method for assessing sleep of a human in a normal sleep environment. The method comprises, or consists essentially of, (a) detecting audible sounds from the human in the normal sleep environment using a mobile electronic device having a sound sensor, and (b) determining whether the audible sounds are indicative of normal sleep or a disorder present in the human. The normal sleep environment can be the bedroom of the human. The audible sounds can comprise snoring sounds of the human. The audible sounds can comprise sounds of the human moving. The audible sounds can comprise sounds of the human sleep talking. The audible sounds can comprise teeth grinding sounds of the human. The mobile device can be a cell phone, smart phone, or an internet connected mobile device. The mobile device can be a personal digital assistant. The audible sounds can comprise snoring sounds of the human, and the determining step can comprise determining that the audible sounds are indicative of normal sleep. The method can comprise informing the human via the mobile device that the human has normal sleep. The audible sounds can comprise snoring sounds of the human, and the determining step can comprise determining that the audible sounds are indicative of the disorder. The disorder can be a sleep disorder. The disorder can be sleep apnea. The disorder can be asthma, chronic obstructive pulmonary disease, or pneumonia. The method can comprise informing the human via the mobile device that the human has the disorder. The audible sounds can comprise snoring sounds of the human, and the determining step can comprise determining that the audible sounds are indicative of sleep apnea. The method can comprise informing the human via the mobile device that the human has sleep apnea. The audible sounds can comprise snores of the human, and the determining step can comprise assessing the amplitude of the snores, the interval between the snores, the frequency composition of snores, or the duration of snores. The audible sounds can comprise snores of the human, and the determining step can comprise assessing the amplitude of the snores, the interval between the snores, the frequency composition of snores, and the duration of snores. The method can comprise using waveform autocorrelation, frequency analysis for identifying an increased variation and power spectrum shift towards higher frequencies, an analysis of cesptral coefficients, or a hidden markov model. The method can comprise recording the audible sounds. The method can comprise recording the audible sounds with the mobile device. The method can comprise transmitting the audible sounds with the mobile device to a computer. The method can comprise recording the transmitted audible sounds on recordable medium. The determining step can comprise obtaining a Fourier transform of at least a segment of the audible sounds. The method can comprise detecting the audible sounds in stereo using the sound sensor and a second sound sensor. The second sound sensor can be connected to the mobile device with wires. The second sound sensor can be connected wirelessly to the mobile device. The second sound sensor can be within a second mobile device. The method can comprise detecting video signals from the human. The method can comprise using one or more sensors to measure oxygen saturation, breathing, heart rate, electrocardiographic information, posture, body movements, electroencephalographic information, nasal air flow, oral air flow, CO₂ levels, body temperature, air temperature, or bioimpedance. The one or more sensors can be attached to the human. The determining step can be performed by an electronic computing device programmed to analyze data from a digital representation of the detected audible sounds. The electronic computing device can be part of a unitary structure with the mobile electronic device.

In another aspect, this document features a method for assessing a human for a likelihood of obstructive sleep apnea in a normal sleep environment. The method comprises, or consists essentially of, (a) obtaining clinical information about the human, (b) detecting audible sounds from the human in the normal sleep environment using a mobile electronic device having a sound sensor, and (c) determining whether the human is likely to experience obstructive sleep apnea based on the clinical information and the audible sounds.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 shows a sleep apnea detection system in accordance with some embodiments.

FIG. 2 shows an exemplary algorithm for a mobile device application for detection, analysis, and display of snoring information.

FIG. 3 shows an exemplary system where a user can incorporate multiple physiologic data inputs into an analysis algorithm. The multiple inputs could be generated by a body worn sensor system.

FIG. 4 shows a variety of outputs for an exemplary sleep/snoring algorithm.

FIG. 5 shows audio tracings of both apneic and simple snoring. The envelope, or tracing over the audio waves shows sharper peaks and more sporadiac peaks during apneic snoring. A fast fourier transform (or other forms of analysis) of the audio signals would show a great variety of frequencies and higher power for apneic snoring.

FIG. 6 is shows an exemplary iterative approach for analyzing a patient.

Like reference symbols in the various drawings indicate like elements.

### DETAILED DESCRIPTION

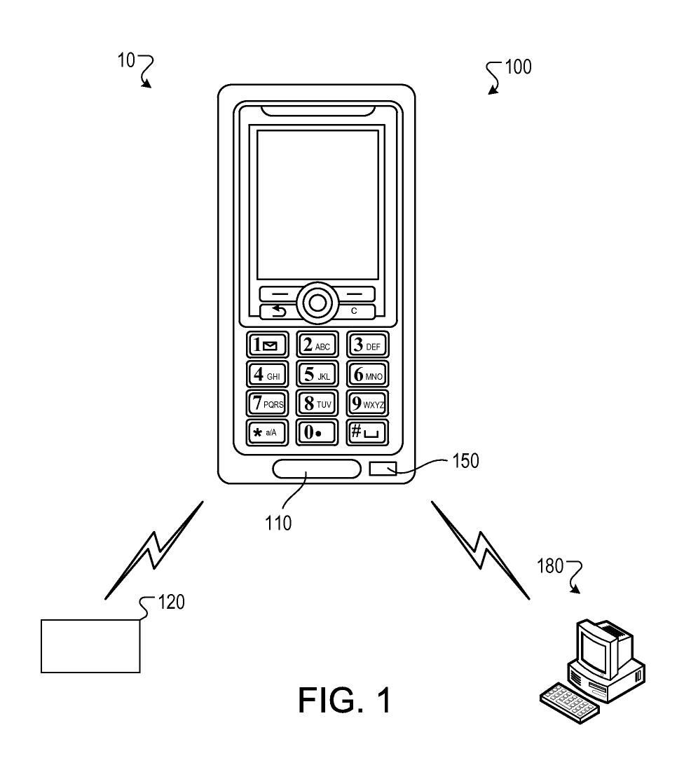

Referring now to FIG. 1, in some embodiments, a sleep apnea detection system 10 includes a mobile device 100 (e.g., a cell phone, smart phone, iPhone, iPod, PDA, and the like) and an application 150 (e.g., software) adapted to run on the mobile device 100 that can store and analyze data for the purpose of determining if an individual is suffering from a disorder such as sleep apnea. For example, a subject that is believed to be suffering from a disorder, such as sleep apnea, can set up the mobile device 100 in a bedroom or other sleeping environment and can allow the mobile device 100 to record data while the subject sleeps. The application 150 can register variables related to a user such as sounds (e.g., for the purpose of determining snoring), oxygen saturation level, heart rate, EKG, breathing frequency, movement, posture, EEG, EOG, video, and the like. By analyzing this data, the application 150 or another application to which this data is transferred, can determine if a user is suffering from a disorder such as sleep apnea. Data stored during the sampling period and subsequent analysis data can be stored on a recordable medium, transferred to another computing device, and the like. For example, data stored by the mobile device can be transferred to a remote computer for analysis. After analysis, whether performed by the mobile device 100 or a remote analysis device, the mobile device 100 can inform the user (or a medical professional) if there are indications of, for example, simple snoring, apneic snoring, sleep talking, sleep walking, or other disorders or indications that the analysis was performed successfully and no abnormal behavior was detected. In some cases, the mobile device can transfer information to a local computer, or to a server via a direct internet connection wirelessly, requiring no active participation of the user. In some cases, the computer or server can generate reports of the sleep data and analysis that can in a controllable manner be shared with the user, the user's physician, or other user selected delegates (e.g., family members).

In some cases, a dedicated sessile device can be used in place of or in addition to a mobile device. For example, dedicated sessile device can be a more permanent fixture at the bedside.

In some examples, application 150 can enable the mobile device 100 to record the sounds picked up from a microphone 110 included in the mobile device 100 and analyze the recorded sounds to determine if the user is suffering from sleep apnea. In some embodiments, in use, the application 150 can cause the mobile device 100 to instruct a user to make some sounds to calibrate the application 150. For example, the mobile device can instruct the user to count out load (e.g., to say “one . . . two . . . three . . . ”), replicate a snore, speak a prompted phrase, provide other spoken sounds, and the like. This can provide sounds to the application 150 that can be used to determine a baseline decibel level and individual tonal qualities to distinguish the user from other individuals who may be sleeping in the same room. This could, for example, be used to estimate a distance between the speaker and the phone, provide information regarding the sonic frequency of the user's speech, and the like. If a replicated snore or pre recorded actual snore is provided by the user for a template after prompting by the software, this can be used to create a template to which future recorded snores can be compared. In some embodiments, replicated snores can be used to provide baseline information such as, resonant frequency, tone, decibel level, and the like, that is specific to the individual user. In some embodiments, the mobile device 100 can receive data from one or more remote sensors (e.g., the remote sensors 120) and can store and/or analyze the data received.

In some cases, in addition to acquiring baseline vocal tonal and frequency qualities, application 150 can prompt the user for useful clinical information. This information can include, without limitation, questions such as height, weight, the presence of hypertension, daytime somnolence, and other questions to provide pertinent clinical information. This information can seed the algorithm and later be used by application 150 in conjunction with sleep sound analysis to determine whether OSA is present. The information may be presented as questions on the screen or spoken questions (with speech generated by the device 100, and answers may be provided by tapping keys or a screen, by speaking to the voice (using voice recognition), or by any manner in which human input may be recognized by a mobile device.

When a user is sleeping, application 150 can cause mobile device 100 to record sounds picked up by microphone 110. During or after this recording, application 150 can analyze these sounds (or transmit sounds for analysis) to determine whether OSA is present. In some cases, it can make comparisons between a digital representation of the recorded data and previously recorded calibration data. For example, comparisons of recorded data to the calibration data can be used to identify when a user emits a snore, when the user speaks (e.g., sleep talking), when a user changes position, and the like. The frequency, volume, and other details about emitted snores can be useful in determining if the user is suffering from sleep apnea. Furthermore, the application 150 can identify sleep-talking events such that these events can be reviewed at a later date. Such a review could be useful to the user in that these events could be used in stress management, psychological therapy, and the like. In some cases, calibration data are not required for sound analysis. Rather, known characteristics of snoring sounds, and apneic snoring in particular, are graded to determine whether or not OSA is present.

In some embodiments, the application 150 can process recorded data to determine information about snoring that took place during the recording session. For example, the application 150 can identify when snoring events occur, the loudness of the snoring events, the intervals between snoring events, and the percentage of the session spent snoring. In another example, application 150 can determine the nature of snoring events such as inspiratory gasping as compared to regular snoring with short intervals between snores. This algorithm used to identify each sound type will be described in greater detail below. The duration of apnea can be assessed by examining, for example, the amplitude of the audio of snoring events, the intervals between snoring events, the frequency of snoring events, the duration of snoring events, the pattern of snoring events, the nature of the snoring events, and the like. In this way, an individual or the application 150 itself can determine if snoring events did occur, the type of events, how loud the snoring was compared to reference noise or a user template, and the like. It is recognized that the interval between audible breathing and a terminating snort (the sound event that completes an apnea) is an interval that may reflect the duration of an apnea event. A determination of the number of apneic events in an hour can be clinically useful, and is termed the apnea hypopnea index (AHI). The above analysis can be designed to permit non-invasive assessment of sleep, including an estimation of AHI and other parameters as currently used in clinical polysomnography. In some embodiments, data recorded by the mobile device 100 can be transmitted to separate analysis device (e.g., a computing workstation 180)

As described herein, application 150 can cause mobile device 100 to record sounds detected using microphone 110. Microphone 110 can also receive sounds other than snoring events. For example, mobile device 100 can record other relevant acoustic parameters such as, ambient noise, noises from pets, talking during sleep (self and spouse), bruxism (teeth clenching or grinding), and the like. These sounds could be utilized to determine the cause of sleep disturbances.

In some embodiments, the use of “stereo” sound analysis can be used to determine placement of the monitoring mobile device 100. For example, utilization of two or more microphones can allow for the comparison of incoming sounds. The multiple microphones can be part of a single mobile device 100 (e.g., some mobile devices 100 may include multiple microphones, additional remote microphones can be controlled by the mobile device 100, and the like), or alternatively, multiple mobile devices can be utilized, each providing one or more microphones, such that the recorded sounds can be recorded simultaneously by a single mobile device 100, can be synchronized at a later time, and the like. In some cases, when sound inputs arrive at equal times and intensities at different microphones, it may be determined then that the device is positioned correctly/optimally. Alternatively the microphone(s) can move to localize sound events and/or optimize signal strength. Use of stereo analysis to localize sound can assist in identifying the location of sounds to be monitored and to eliminate sounds from locations known to not be associated with the location of the sounds to be monitored. In some cases, if the position of the sound receivers is known, the distance to the site of the sound of interest can be triangulated by calculating the difference in time between sound arrival at each recording site.

In some cases, a microphone can be mounted in close proximity to the nose (e.g., adhered to the upper lip or attached to the nares) or mouth. The use of a microphone with added capacity to detect airflow (flow sensitive) can add to the accuracy of identifying apneic snoring.

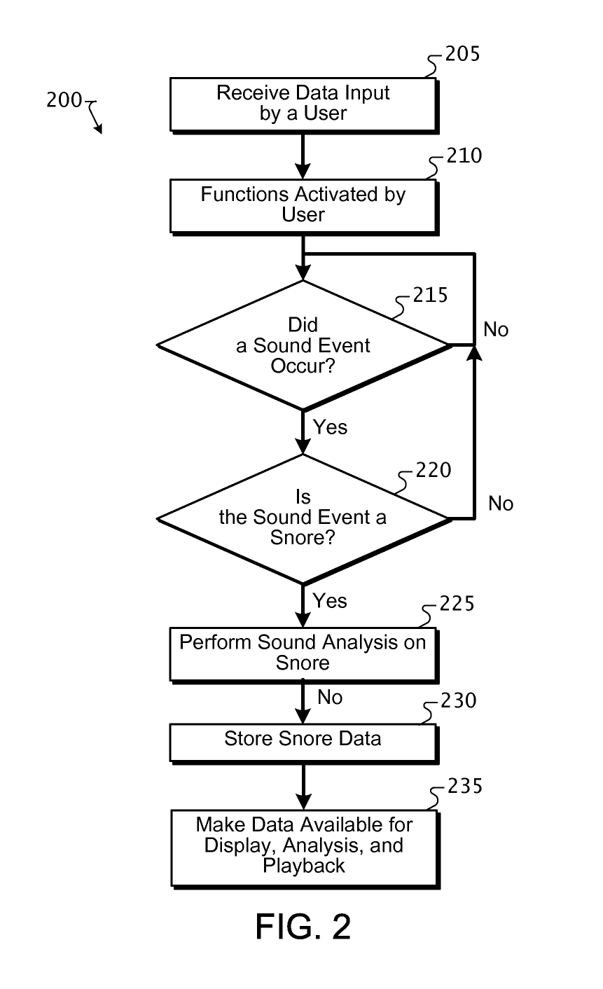

Referring in more detail to the illustrative process 200 shown in FIG. 2, the process 200 for diagnosing sleep apnea can be performed by a mobile device 100. In operation 205, the mobile device 100 can receive data input by a user, which can be used later by the sleep apnea detection system 10 to determine if a subject suffers from a pathologic sleep disorder such as sleep apnea. Exemplary data that can be input into the mobile device 100 includes the responses to questions from a questionnaire (e.g., a Berlin questionnaire) about the subject, such as height, weight, presence of hypertension, alcohol usage, family history, and the like. The user can also input exemplary audio data by counting out load (e.g., speaking “one . . . two . . . three . . . ”), replicating a snore, speaking a prompted phrase, providing other spoken sounds, and the like. In some cases, a subject can be asked to whisper and be prompted to reposition the recording device if a quiet whisper lacks sufficient intensity for proper sound analysis. In some cases, a whisper can be a specific word or phrase (e.g., “test”), and the ability of the system to identify the anticipated phrase can determine whether the location and position of the device permits suitable quality recording. As described herein, these audio data can provide sounds to system 10 that can be used to determine a baseline decibel level, create a template to which future recorded snores can be compared, provide baseline information, and the like.

In operation 210, system 10 can be activated by a user to begin monitoring a subject for sound events. In operation 215, if a sound event does occur, process 200 can proceed to operation 220, otherwise, process 200 can remain at operation 215 until a sound event does occur. In operation 215, a bandpass filter can be applied to the recorded sound to permit only analysis of sound in the frequency range of human snores. This filter can eliminate adventitial sounds, lowering the likelihood of false positive snores by only submitting to analysis sounds in the snoring frequency. In some cases, the expected bandpass filters can be pre-specified or can be modified for clinical information (e.g., age, gender, etc.) or for verbal cues for baseline. In operation 220, process 200 can determine if the sound event was a snore. This can be performed by the mobile device 100 and can be determined by algorithms, comparisons to templates, and the like. If the sound event is determined to be a snore, the process can proceed to operation 225. Otherwise, the process 200 can return to operation 215 and wait for the next sound event. In operation 225, the system 10 can perform additional sound analysis on the snore determined in operation 220 to determine if the snore falls into the category of physiologic (e.g., simple) snoring or pathologic (e.g., apneic) snoring. This can be performed by the mobile device 100 and can be determined by algorithms, comparisons to templates, and the like. In operation 230, data associated with the snore can be stored. For example, the audio and the results of the previous analysis can be stored. In operation 235, the data stored in operation 230 can be made available for playback, display, further analysis, and the like.

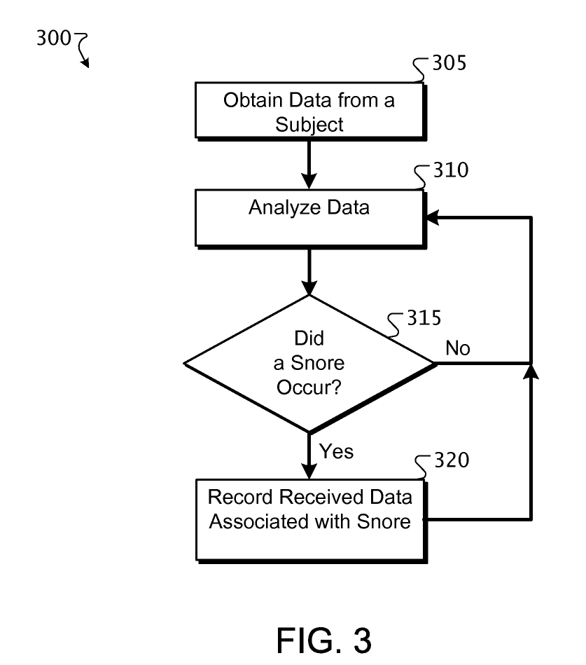

Referring in more detail to the illustrative process 300 shown in FIG. 3, the process 300 for diagnosing sleep apnea can be performed by components of a sleep apnea detection system 10 (e.g., a mobile device 100, a computer workstation, and the like). In operation 305, a component of a sleep apnea detection system 10 can obtain data from a subject that may have been obtained from one or more sensors. These data can be used by the sleep apnea detection system 10 to determine if a subject suffers from a pathologic sleep disorder such as sleep apnea. For example, the mobile device 100 can receive data from one or more sensors and can perform the subsequent steps of the process 300 described herein. In another example, data recorded by the mobile device 100 can be transferred to an analysis device (e.g., a computing workstation) during operation 305 such that the analysis device performs the subsequent steps of the process 300 described herein. Exemplary data that can be obtained by the system 10 includes audio data from one or more microphones (e.g., internal or external to the mobile device 100), video, accelerometer data, ECG data, blood oxygen saturation data, and the like.

In operation 310, the system 10 can perform an analysis on a portion (e.g., a suspected snore event) of the data obtained to determine if the data is indicative of a snoring event. This can be performed by the mobile device 100 or another analysis device and can be determined by algorithms, comparisons to templates, and the like. If, in operation 315, it is determined that the portion of data analyzed is not indicative of a snore event, the process 300 can return to operation 310 to analyze additional data, if present. If it is determined that a snore event did occur, in operation 320 the data associated with the snore (including data generated during operation 315) can be stored for future playback, display, further analysis, and the like. For example, the data can be recorded on a recordable medium such as a USB drive. In some cases, it can be stored directly on the mobile device, or transmitted wirelessly to a server (“the cloud”) to permit access and review from mobile or Internet connected computers with appropriate security access.

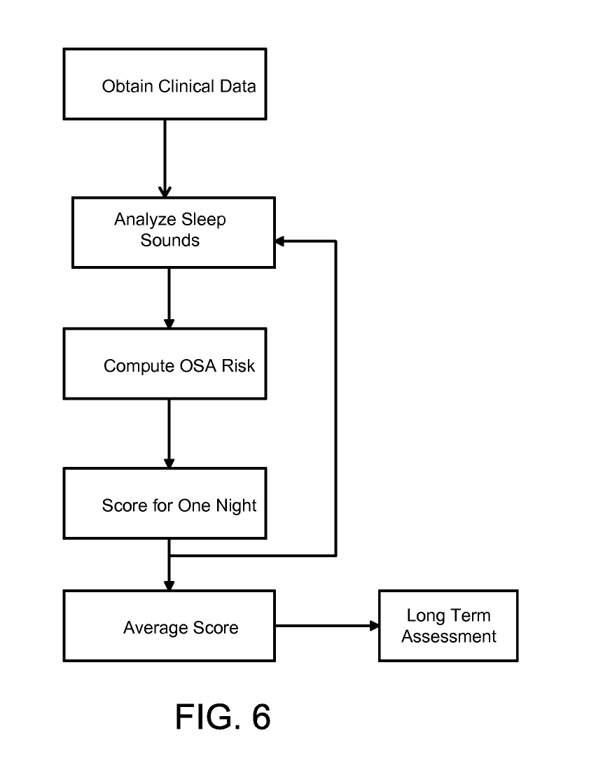

In some cases, the methods and materials provided herein can be used to calculate (e.g., algorithmically calculate) an OSA risk score based on clinical data, sleep sound analysis, or both clinical data and sleep sound analysis (FIG. 6). In some cases, an integrative approach can be used to diagnose sleep apnea or calculate an OSA risk score. For example, data can be obtained over multiple nights (e.g., 2, 3, 4, 5, 10, 15, 20, 30, 40, 50, or more nights), and additional OSA burden analysis and trending can be performed over time.

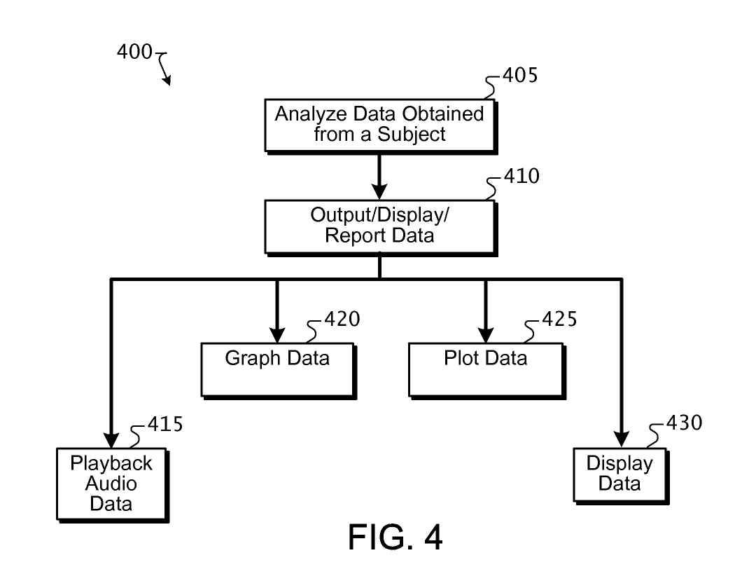

Referring in more detail to the illustrative process 400 shown in FIG. 4, the process 400 to present data and algorithmic results to facilitate a physician's diagnosing sleep apnea can be performed by components of a sleep apnea detection system 10 (e.g., a mobile device 100, a computer workstation, and the like). In operation 405, a component of a sleep apnea detection system 10 can perform an analysis on a portion of data obtained from a subject to determine if the data are indicative of a pathological sleep disorder such as sleep apnea. This can be performed by the mobile device 100 or another analysis device and can be determined by algorithms, comparisons to templates, and the like. In operation 410, the data obtained from the subject and results of the analysis can be output by subsequent steps of the process 400.

For example, operation 415 can cause the system 100 to playback at least a portion of audio data obtained from a subject. The playback can be of time periods that have been determined to be indicative of snoring events, time periods containing unknown sounds, time periods indicative of apneic snoring events, all sounds recorded, and the like. In operation 420, the process 400 can cause graphs to be output. For example, the graphs can depict sound intensities, Fourier transforms, and the like. These graphs can be of time periods that have been determined to be indicative of snoring events, time periods containing unknown sounds, time periods indicative of apneic snoring events, all times, and the like. These graphs can be displayed on a mobile device, or via an internet connected computer with appropriate access. In operation 425, the process 400 can cause plots to be output. For example, the plots can depict the frequency of apneic and simple snoring events, the duration of apneic and simple snoring events, the sonic intensity of apneic and simple snoring events, inter-snoring cycle length, the total number of cycles over the total sampled period, and the like. In operation 440, the process 400 can cause data to be output. For example, the oxygen saturation levels can be output (e.g., during snoring vs. non-snoring periods), snoring events can be output along with body position, and the like. When multiple on and off body sensors are used, their outputs can be simultaneously displayed to demonstrate interrelationships of the physiologic parameters recorded.

In some cases, a sleep apnea detection system provided herein can be configured to perform a “quality assessment” of audible signals or a recording. For example, if no normal breathing is detected or recorded, the device may have been covered or moved too far away from the sleeper. In such cases, an alert can be provided, and/or the detected signals can be removed from further analysis. In some cases, a quality assessment can be performed to assess the device for inadvertent power downs, to assess the audible signals or recordings for a sufficient duration, and/or to assess the audible signals or recordings for excessive noise that is clearly not physiologic.

In some cases, summary information can be “packaged” and sent to the subject, a physician, or family member with degrees of detail transmitted optionally controlled by the user. In some cases, if sleep apnea is detected, an automatic signal can be sent to the user or patient to abort the apnea or activate a device to ameliorate the apnea (e.g., a positive airway pressure device). For example, a device provided herein can be configured to provide a signal to activate a positive airway pressure device at those times when sleep apnea is detected so as to provide the user with intermittent positive pressure as needed. In some cases, a device provided herein can be configured to activate a device (e.g., a wearable device) such that the activated device provides a stimulus (e.g., an audio, tactile, buzz, or electric stimulus) to the user designed to stimulate breathing.

### Snoring Algorithms

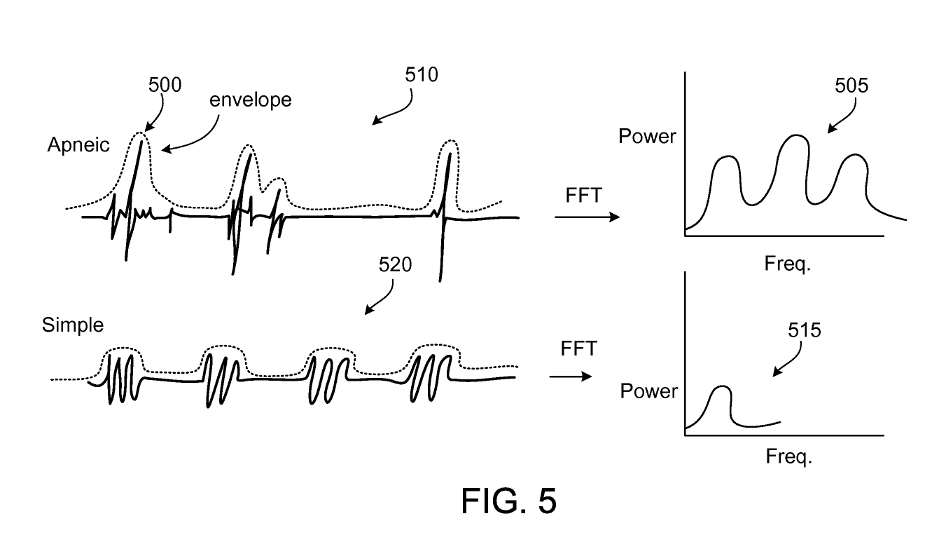

As snores were examined, one of the unique characteristics identified is that there can be a repetitive pattern. One exemplary estimate is that there are as many as 3-10 rapid repeats in an interval of 0.4 to 2 seconds. This characteristic pattern, which can be caused by air squeaking through a small space (with associated Bernoulli-like pressure differentials) that is bounded by flaccid soft tissues permitting their vibration, can be used for sound identification and differentiation. In one example, rapid sound repetition can be used as a means for distinguishing a snoring event from other ambient sounds including speech, which would lack that type of repetition. One method for performing this differentiation is an assessment of autocorrelation of the envelope of the recorded sound. This could be at a much higher frequency than the rhythmic repetition of normal non-apneic snoring. (e.g., with a snore every few seconds, without the differentiating “vibrato” components, and the like). In addition to a very rapid envelope autocorrelation, a simple power spectrum may show both an increased variation in power spectrum during apneic snores compared to baseline and a high energy density in the high frequency band (e.g., in the frequency range of the vibration) during snoring versus other types of sounds (See FIG. 5).

In some embodiments, the rapid repetitive nature of the sounds permits facilitated detection using standard speech recognition technology. Thus, methods known in the art (e.g., dynamic time warping) can be used to identify normal breathing and pathologic breathing, as well as sleep talking. Other exemplary methods, such as vocal tract length normalization (e.g., used to distinguish male/female speakers) and maximum likelihood linear regression can be applied. In some embodiments, these methods can be used in conjunction with a known speech sample at the beginning of the recording (e.g., as described previously in conjunction with the creation of sound templates). Another exemplary method involves using information collected from the subject (e.g., height, weight, gender, and the like) to predicatively determine the vocal range expected and increase yield.

In some embodiments, with regard to detection of the rapid, repetitive, and vibrating sound, speech detection mathematics can be applied. For example, one approach would be to take a Fourier transform of a short segment and to decorrelate it using a cosine transform, to obtain the cepstral coefficients. These were initially developed to look at seismic echoes from earthquakes and explosions and thus can be used to examine the reverberations associated with snoring. The first few coefficients from the cosine transform can be fed into a hidden Markov model to determine the probability of the sound under evaluation being a snore event and the type of snore event. This approach can be utilized in detection of snoring, wheezing, gasping, etc. in various disease detection algorithms. While this embodiment and disclosure is focused on sleep apnea, it is recognized that talking or vocalization during sleep, asthma, COPD, pneumonia, croup, infantile apnea, and other conditions with associated audible characteristics can be evaluated as described herein.

In some embodiments, snoring can be the loudest sound generated during the night. Detection can start with identification of sounds above a decibel threshold or the loudest sounds occurring over a period of time. These sounds can then be detected, tracked, recorded, and analyzed. Given that there can be non-sleep sounds present in a room, as noted above, a bandpass filter can be applied before thresholding increasing the likelihood that the sound evaluated will be snore-related.

### Detection Algorithms

There can be several primary areas of sound wave characteristics, examples of which can include the amplitude of the snores, the interval between snores, the frequency composition of each snore, the duration of each snore, and the like. When comparing pathologic (e.g., apneic) snoring sounds to physiologic sounds (e.g., simple snoring), the variability of each of these characteristics can be increased. In other words, compared to simple snoring, apneic snoring can include intervals between sounds with greater variability, amplitudes within individual snore events with greater variability, amplitudes of individual spikes within each snore event with greater variability, and durations (in milliseconds) of the sound waves clumped together during each snore event with greater variability. Additionally, there is a shift in the power spectrum toward different frequencies in the setting of pathologic snoring. These audio characteristics can be combined with other sensed information such as airflow, oxygen saturation, etc. and integrated into the algorithm to improve sensitivity and specificity of detecting and distinguishing apneas.

There can be high variability in the frequency of the snoring sounds at their end point, and apneic episodes can include a higher frequency component. Thus, exemplary methods for characterizing pathologic snoring sounds from physiologic sounds can include looking at the overall standard deviation or variation of the spectrum of a snore, assessing the amount of energy in a high frequency end of that component, assessing the ratio of high frequency to low frequency content in a signal that is already identified as a snore event, and the like. For example, a shift in the power spectrum from low to high frequency may be characteristic of pathologic snoring. In some embodiments, a score that identifies, for example, an increase in standard deviation, another measure of variability (e.g. the integral of the individual sound wave frequencies that make up the snore to give us a measure of variability), and the like, can be used, either in addition to or in lieu of a measure of power. Using a scoring system as described can differentiate between simple snoring and apneic snoring. In some examples, creating an “envelope” (e.g., an envelope 500 as depicted in FIG. 5) of the sound waves for each type of snoring can be used for autocorrelation and for representation of the variability in amplitude and frequency of sounds for each type of snoring used by the algorithm (see FIG. 5).

TABLE 1 includes exemplary audio differentiators of simple versus apneic snoring. Other differentiators that can be measured by other sensors and included or integrated into an algorithm include, without limitation, absence of airflow, decrease in oxygen saturation, visible “suffocation,” and increase in blood pressure and heart rate.

TABLE 1
Exemplary Differentiators of Apneic Versus Simple Snoring

| Apneic Snoring | Simple Snoring |
|---|---|
| Rapid rise in sound intensity | Long, slow rise of sound (smaller |
| | slope in time domain) |
| Burst pattern, multi-fragmented | Rhythmic sounds |
| sounds over a short period | |
| Clustered events, often occurring | Occur for long periods of time and |
| during supine positioning time and | for a large percentage of total sleep |
| REM sleep. Longer durations of | |
| apneic events can be worse. | |
| Fast-Fourier transform of signal | Fast-Fourier transform of signal |
| can show a complex, multi-modal | can show a less complex pattern |
| frequency spectrum with higher | with lower dominant frequency |
| energy in the high frequency range. | and fewer peaks |
| Silence followed by a snort and/or | Rhythmic snoring. |
| gasp | |

In some embodiments, time duration differences can be identified to help distinguish between apneic snoring and simple snoring with patient movement (e.g. simple snoring plus rolling over or changing body position which could result in variability in sound frequency and/or intensity). Simple snoring plus patient motion can cause sound signal variability over a relatively short period of time (e.g., about 30 seconds) whereas apneic snoring can produce sound frequency variability for longer periods of time (e.g., about 1-4 minutes, about 20 minutes, and the like).

In some embodiments, variability analysis can be performed over, for example, 4 minutes. While the total variability of these measures over 4 minutes can be important, the variability can be broken down into individual minutes. High variability over each individual minute can be indicative of apneic snoring. However, if the variability for minute 1 is low, for minute 2 is high, and for minute 3 and 4 are low, this can be indicative of, for example, the subject changing position to be further away from the microphone, thus indicating that this may not be an apneic snoring event even though the variability for the overall 4 minutes may be high. Thus, trending of the apnea risk over time can be used within a single night to assess the likelihood of the presence of pathologic OSA.

In looking at a Fourier transformation of sound waves, apneic snoring can include a wide range of different sound waves that make up a single snore event (see FIG. 5). Similarly when multiple snores are superimposed, the variation of different frequencies that comprise the snores can be high. In terms of quantifying this, the integral of the Fourier transform 505 of an apneic snoring event 510 can be compared to the Fourier transform 515 of a simple snoring event 520, which can be used to quantify the measurements of apneic snoring as compared to simple snoring. In this example, while the loudness of sounds may change, the frequency distribution may not change. Thus, the wide range of frequencies that comprise apneic snoring can be evident in the Fourier transform even though the subject may change posture.

In some embodiments, a cutoff or weighting of the number of frequencies in each snore can be used to differentiate between an apneic snore and a simple snore. With simple snoring, there can be a finite number of different frequencies constituting the simple snore whereas apneic snoring can include a greater number of frequencies constituting the snoring.

### Multi-Physiologic Inputs and Measurements

In some embodiments, one or more customized monitoring strips or devices can be placed over, for example, the chest wall, forehead, leg, arm, and the like, to transmit data to the mobile device. Exemplary data that can be transmitted (e.g., wirelessly transmitted or transmitted via wires) using these monitoring strips can include oxygen saturation, accelerometer measured monitored breathing, accelerometer measured heart rate, EKG, accelerometer measured posture (e.g., to determine number of times patient gets out of bed, sleep walks, and the like), accelerometer measured movement (e.g., to determine sleep posture, when the user changes position, phenomena such as restless sleep, and restless legs, parasomnias and REM related sleep disorders, and the like), EEG (e.g., to monitor sleep, sleep stage, and wakefulness), nasal and/or oral air flow (e.g., including a CO2 monitor), temperature monitoring (e.g., on body or in air sensor), CO2 monitoring (e.g., by expired air and/or transcutaneously), bioimpedance (e.g., respiration or heart rate), sonic data (e.g., using a microphone) (see FIG. 3). In these embodiments, continuous monitoring can be achieved by using, for example, a mobile device placed in proximity to the subject and one or more disposable or nondisposable strips or monitors customized for monitoring variables that are relevant to a particular patients need. Advantageously, the disposable strips could be obtained over-the-counter purchase and such a system can obtain one or more days of monitoring. Also advantageously, transmitting oxygen saturation over a mobile device can provide the same functionality, with greater ease of use, as compared to the traditional overnight oximetry.

In some embodiments, the detection of sound waves indicative of wheezing or chest congestion can be used along with the sounds waves of exhalation to identify prolongation of expiration and to detect asthma or other respiratory conditions. Use of an accelerometer in contact with the patient can be utilized in exemplary ways such as to eliminate ambient/contaminating noise associated with patient movement, to detect respiratory motion and/or sounds (talking, bruxism, etc.), and the like. This signal can be used to verify and/or back-up the detection taking place on the mobile device. For example, accelerometer data can be used to determine if the users face is pointing towards the mobile device or away, thus indicating to make adjustments in signal gain. In some cases, such a system can be used to monitor the breathing of infants when apnea is a concern. A significant apnea can result in an audible alert to arouse the infant, other electrical or vibratory stimulation of the infant as well as an immediate parental alert via electronic means (e.g., SMS/text, cell call, house phone call) by the monitoring system.

In some embodiments, a device worn by the patient can also be used to determine relative distance and position between the patient and the mobile device. For example, by determining this distance, the system can allow for signal amplification if necessary and gradations of signal amplification as the distance increases. Sleep disorders (e.g., snoring, sleep walking, REM related behavioral disorders, etc.) can also be detected to trigger video capture.

In some cases, a throat microphone or accelerometer (e.g., a wireless throat microphone or accelerometer) can be used to reduce the impact of other noises on the recording and the impact of different sleep positions. Depending upon the design of the microphone/accelerometer, the device can be immune to background noise. The signals can be normalized, because of the proximity to the source of sleep sounds.

A number of embodiments of the invention have been described. Nevertheless, it will be understood that various modifications may be made without departing from the spirit and scope of the invention. Accordingly, other embodiments are within the scope of the following claims.

1. A method for assessing sleep of a human in a normal sleep environment, wherein said method comprises:
(a) detecting audible sounds from said human in said normal sleep environment using a mobile electronic device having a sound sensor, and
(b) determining whether said audible sounds are indicative of normal sleep or a disorder present in said human.

2-8. (canceled)

9. The method of claim 1, wherein said audible sounds comprise snoring sounds of said human, and said determining step comprises determining that said audible sounds are indicative of normal sleep.

10. (canceled)

11. The method of claim 1, wherein said audible sounds comprise snoring sounds of said human, and said determining step comprises determining that said audible sounds are indicative of said disorder.

12-14. (canceled)

15. The method of claim 11, wherein said method comprises informing said human via said mobile device that said human has said disorder.

16. The method of claim 1, wherein said audible sounds comprise snoring sounds of said human, and said determining step comprises determining that said audible sounds are indicative of sleep apnea.

17. The method of claim 16, wherein said method comprises informing said human via said mobile device that said human has sleep apnea.

18. The method of claim 1, wherein said audible sounds comprise snores of said human, and said determining step comprises assessing the amplitude of said snores, the interval between said snores, the frequency composition of snores, or the duration of snores.

19. The method of claim 1, wherein said audible sounds comprise snores of said human, and said determining step comprises assessing the amplitude of said snores, the interval between said snores, the frequency composition of snores, and the duration of snores.

20. The method of claim 1, wherein said method comprises using waveform autocorrelation, frequency analysis for identifying an increased variation and power spectrum shift towards higher frequencies, an analysis of cesptral coefficients, or a hidden markov model.

21. The method of claim 1, wherein said method comprises recording said audible sounds.

22. (canceled)

23. The method of claim 1, wherein said method comprises transmitting said audible sounds with said mobile device to a computer.

24. The method of claim 23, wherein said method comprises recording said transmitted audible sounds on recordable medium.

25. The method of claim 1, wherein said determining step comprises obtaining a Fourier transform of at least a segment of said audible sounds.

26. The method of claim 1, wherein said method comprises detecting said audible sounds in stereo using said sound sensor and a second sound sensor.

27-29. (canceled)

30. The method of claim 1, wherein said method comprises detecting video signals from said human.

31. The method of claim 1, wherein said method comprises using one or more sensors to measure oxygen saturation, breathing, heart rate, electrocardiographic information, posture, body movements, electroencephalographic information, nasal air flow, oral air flow, CO₂ levels, body temperature, air temperature, or bioimpedance.

32. The method of claim 31, wherein said one or more sensors are attached to said human.

33. The method of claim 1, wherein said determining step is performed by an electronic computing device programmed to analyze data from a digital representation of said detected audible sounds.

34. The method of claim 33, wherein said electronic computing device is part of a unitary structure with said mobile electronic device.

35. A method for assessing a human for a likelihood of obstructive sleep apnea in a normal sleep environment, wherein said method comprises:
(a) obtaining clinical information about said human,
(b) detecting audible sounds from said human in said normal sleep environment using a mobile electronic device having a sound sensor, and
(c) determining whether said human is likely to experience obstructive sleep apnea based on said clinical information and said audible sounds.
